(19) United States
(12) Patent Application Publication
(10) Pub. No.: US 2025/0313860 A1
(43) Pub. Date: Oct. 9, 2025

(54) EXPRESSION OF EXOGENOUS PROTEINS IN DONOR PLATELETS TREATED WITH LIPID NANOPARTICLES

(71) Applicant: THE UNIVERSITY OF BRITISH COLUMBIA, Vancouver (CA)

(72) Inventors: Christian J. KASTRUP, Milwaukee, WI (US); Jerry LEUNG, Vancouver (CA); Colton STRONG, Vancouver (CA); Katherine BADIOR, Milwaukee, WI (US); Pieter R. CULLIS, Vancouver (CA); Eric JAN, Vancouver (CA); Madelaine ROBERTSON, Vancouver (CA); Dana DEVINE, Vancouver (CA)

(73) Assignee: THE UNIVERSITY OF BRITISH COLUMBIA, Vancouver, BC (CA)

(21) Appl. No.: 18/866,923

(22) PCT Filed: May 15, 2023
(86) PCT No.: PCT/CA2023/050665

§ 371 Date: Nov. 18, 2024

Related U.S. Application Data
(60) Provisional application No. 63/344,247, filed on May 20, 2022.

(51) Int. Cl. C12N 15/88; C12N 9/02

(52) U.S. Cl. CPC C12N 15/88; C12N 9/0069; C12Y 113/12013

(57) ABSTRACT

The present disclosure provides a novel lipid nanoparticle (LNP) compositions useful for the transfection and expression of exogenous proteins in platelet cells. In particular, wherein the LNP composition comprises mRNA encoding the exogenous protein and a lipid mixture, and the lipid mixture comprises at least one ionizable cationic lipid, at least one helper lipid, a sterol, and at least one polyethylene glycol (PEG)-lipid conjugate. Further provided are use of the LNP compositions for transfection of platelets with messenger RNA encoding an exogenous protein and expression of the exogenous protein in the resulting transfected platelets.

### CROSS-REFERENCE TO RELATED APPLICATIONS

This patent application claims the benefit of priority from U.S. Provisional Patent Application 63/344,247 filed May 20, 2022; the entire contents of which are incorporated herein by reference.

This invention was made with Government support under W81XWH-20-2-0041 awarded by the Medical Research and Development Command. The government has certain rights in the invention.

### FIELD OF THE INVENTION

The present invention discloses novel lipid nanoparticle compositions useful for the transfection and expression of exogenous proteins in platelet cells.

### BACKGROUND

Platelets are an integral component of hemostasis and donor platelets are routinely transfused to restore hemostatic balance in thrombocytopeniar actively bleeding patients. Platelets also carry potential as a cell therapy-based treatment for non-coagulopathic diseases; for example, they directly interact with cancer cells to promote tumour growth and metastasis, an association that can been exploited to develop platelet-based targeted cancer therapeutics². Platelet-derived extracellular vesicles have also been proposed as drug delivery vehicles due to specific organ targeting. Despite crucial roles in both hemostasis and cell-specific interactions, and thus significant potential as a target cellular therapy, there is a paucity of technologies capable of enhancing platelet biochemistry through genetic modification.

Despite being anucleate, platelets actively synthesize proteins both in vivo and once isolated from donor blood, making them an attractive candidate cell for genetic engineering.^{3,4} This pathway presents an attractive target for genetic engineering, to tune platelet function through protein expression. Existing methods of engineering platelets are indirect and involve transfection of hematopoietic stem cell platelet precursors to produce progenitor platelets with modified function. Two approaches include (1) ex vivo production of platelets from stem cells, which is limited by scalability, and (2) in vivo transfection of bone marrow megakaryocytes which is limited by transfection agent accessibility and specificity.^{5,6} Direct approaches of modifying donor platelet functions are even more limited. Viral based transfection strategies are not useful for direct donor platelet transfection since platelets lack a nucleus and the ability to synthesize their own mRNA transcripts from DNA. Physical and chemical transfection approaches may effectively deliver nucleic acid into platelets; however, protein expression remains to be conclusively observed. Chemical transfection methods such as lipofectamine fails to induce sufficient mRNA uptake by platelets and translation is not observed.⁷ Electroporation, a physical transfection approach that induces small pores in the cell plasma membrane to enable cargo uptake, has not reliably demonstrated protein expression from delivered mRNA.⁸

Lipid nanoparticles (LNP) are a flexible and clinically approved platform which enable delivery and intracellular release of genetic material. Despite being anucleate, and thus unamenable to DNA-based transfection technologies, platelets contain all the translational machinery necessary for protein synthesis. Platelets are therefore ideal for modification by mRNA-containing LNP (mRNA-LNP) to enable expression of exogenous protein.

Advancements in lipid nanoparticle (LNP) technology have dramatically improved the delivery efficiency of mRNA into target cells. LNPs are currently used in the FDA approved siRNA therapeutic, Onpattro, and the Pfizer and Moderna COVID-19 vaccines.^{9,10} Genetically engineering donor platelets using lipid nanoparticles and RNA to produce exogenous protein ex vivo will enable the deliberate modulation of platelet function, or the introduction of new functions that will expand platelets as a cell therapy for therapeutic purposes.

### SUMMARY OF THE INVENTION

The present invention relates to a lipid nanoparticle (LNP) composition for expression of exogenous proteins in a population of transfected platelet cells.

In a first aspect, the invention relates to a lipid nanoparticle (LNP) composition for expression of an exogenous protein in a population of transfected platelet cells, wherein the LNP composition comprises mRNA encoding the exogenous protein and a lipid mixture, and the lipid mixture comprises at least one ionizable cationic lipid, at least one helper lipid, a sterol, and at least one polyethylene glycol (PEG)-lipid conjugate.

In some embodiments, the at least one ionizable cationic lipid in the lipid mixture is CL4H6, SM-102, ALC-0315, CL1H6, CL15H6, CL1D6, ALC-0159, or any combination thereof. In other embodiments, the at least one ionizable cationic lipid is CL4H6 or ALC-0315.

In some embodiments, the at least one helper lipid in the lipid mixture is phosphatidylcholine (POPC), 1,2-dioleoyl-sn-glycero-3-phosphocholine (DOPC), 1,2-distearoyl-sn-glycero-3-phosphocholine (DSPC), 1,2-dioleoyl-sn-glycero-3-phosphoethanolamine (DOPE), dioleoyl phosphatidylglycerol (DOPG), egg sphingomyelin (ESM), or any combination thereof. In other embodiments, the at least one helper lipid in the lipid mixture is phosphatidylcholine (POPC) or 1,2-dioleoyl-sn-glycero-3-phosphocholine (DOPC).

In some embodiments, the at least one ionizable cationic lipid in the lipid mixture is CL4H6 and the helper lipid is phosphatidylcholine (POPC). In other embodiments, the at least one ionizable cationic lipid in the lipid mixture is ALC-0315 and the helper lipid is 1,2-dioleoyl-sn-glycero-3-phosphocholine (DOPC).

In some embodiments, the sterol is cholesterol or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol, or any mixture thereof. In other embodiments, the sterol is cholesterol.

In some embodiments, the at least one polyethylene glycol (PEG)-lipid conjugate is 1,2-dimyristoyl-rac-glycero-3-methoxypolyethylene glycol-2000 (DMG-PEG2000), 1,2-distearoyl-sn-glycero-3-phosphoethanolamine-N-amino(polyethylene glycol)-2000 (DSPE-PEG2000), or PEG-1,2-distearoyl-rac-glycero-3-methylpolyoxyethylene 2000 (DSG-PEG). In other embodiments, the at least one polyethylene glycol (PEG)-lipid conjugate is 1,2-dimyristoyl-rac-glycero-3-methoxypolyethylene glycol-2000 (DMG-PEG2000).

In another aspect, the invention relates to a lipid nanoparticle (LNP) composition for expression of an exogenous protein in a population of transfected platelet cells, wherein the LNP composition comprises messenger RNA (mRNA) encoding the exogenous protein and a lipid mixture, wherein the lipid mixture comprises 30-55 mole percent of the at least one ionizable cationic lipid, 5-20 mole percent of the at least one helper lipid, 25-50 mole percent of a sterol; and 0.5-3 mole percent of the at least one polyethylene glycol (PEG)-lipid conjugate.

In one embodiment, the lipid mixture comprises 50 mole percent of the at least one ionizable cationic lipid, 10 mole percent of the at least one helper lipid, 38.5 mole percent of a sterol; and 1.5 mole percent of the at least one polyethylene glycol (PEG)-lipid conjugate. For example, the lipid mixture may comprise 50 mole percent CL4H6 or ALC-0315 ionizable cationic lipid, 10 mole percent of phosphatidylcholine (POPC) or 1,2-dioleoyl-sn-glycero-3-phosphocholine (DOPC) helper lipid, 38.5 mole percent of cholesterol; and 1.5 mole percent of 1,2-dimyristoyl-rac-glycero-3-methoxypolyethylene glycol-2000 (DMG-PEG2000).

In yet another aspect, the messenger RNA present in the LNP composition may comprise one or more nucleotide modifications. In some embodiments, the modified nucleotide is 5-methoxyuridine, pseudouridine, or N1-methylpseudouridine.

In some embodiments, the mRNA in the LNP composition may encode an exogenous protein such as: a coagulation factor, an anti-fibrinolytic protein, an anticoagulant, a fibrinolytic protein, an antimicrobial protein, an immunomodulator, an anti-cancer protein, a genetic editing protein, or a reporter protein. In other embodiments, the mRNA in the LNP composition is a non-coding RNA or DNA aptamer.

In another aspect, the present invention relates to a lipid nanoparticle (LNP) composition for expression of an exogenous protein in a population of transfected platelet cells, wherein the LNP composition comprises mRNA encoding the exogenous protein and a lipid mixture, the lipid mixture comprising at least one ionizable cationic lipid, at least one helper lipid, a sterol, and at least one polyethylene glycol (PEG)-lipid conjugate, wherein the mRNA encoding the exogenous protein may comprise one or more modified nucleotides. For example, the mRNA encoding the exogenous protein may comprise 5-methoxyuridine, pseudouridine, or N1-methylpseudouridine.

In still another aspect, the present invention relates to the use of the above LNP compositions for transfection of platelets with messenger RNA encoding an exogenous protein and expression of the exogenous protein in the resulting transfected platelets.

In some embodiments, the present invention relates to use of an LNP composition wherein the ionizable cationic lipid in the lipid mixture is CL4H6 and the helper lipid is 1,2-dioleoyl-sn-glycero-3-phosphocholine (DOPC). When such LNP composition is used to produce a population of transfected platelet cells expressing an exogenous protein, such population of transfected platelet cells exhibits only minimal activation, as evidenced by cell surface exposure of platelet activation marker CD62P at levels less than those seen in platelets prepared in parallel with transfected platelets, but receiving 0.1 U mL⁻¹ thrombin in lieu of LNP.

In a final aspect, the present invention relates to a population of platelets expressing an exogenous protein, wherein said population of platelets is transfected with an LNP composition comprising a mRNA encoding the exogenous protein and a lipid mixture, wherein the lipid mixture comprises at least one ionizable cationic lipid, at least one helper lipid, one or more sterol; and at least one polyethylene glycol (PEG)-lipid conjugate.

In some embodiments, the population of platelets is transfected with an LNP composition comprising a mRNA encoding an exogenous protein and a lipid mixture, wherein the at least one ionizable cationic lipid in the lipid mixture is CL4H6, SM-102, ALC-0315, CL1H6, CL15H6, CL1D6, ALC-0159, or any combination thereof. For example, the one ionizable cationic lipid is CL4H6 or ALC-0315.

In some embodiments, the population of platelets is transfected with an LNP composition comprising a mRNA encoding the exogenous protein and a lipid mixture, wherein the at least one helper lipid in the lipid mixture phosphatidylcholine (POPC), 1,2-dioleoyl-sn-glycero-3-phosphocholine (DOPC), 1,2-distearoyl-sn-glycero-3-phosphocholine (DSPC), 1,2-dioleoyl-sn-glycero-3-phosphoethanolamine (DOPE), dioleoyl phosphatidylglycerol (DOPG), egg sphingomyelin (ESM), or any combination thereof. In other embodiments, the population of platelets is transfected with an LNP composition comprising a mRNA encoding the exogenous protein and a lipid mixture, wherein the at least one helper lipid in the lipid mixture is phosphatidylcholine (POPC) or 1,2-dioleoyl-sn-glycero-3-phosphocholine (DOPC).

In some embodiments, the population of platelets is transfected with an LNP composition comprising a mRNA encoding the exogenous protein and a lipid mixture, wherein the sterol in the lipid mixture is cholesterol or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol, or any mixture thereof. In other embodiments, the population of platelets is transfected with an LNP composition comprising a mRNA encoding the exogenous protein and a lipid mixture, wherein the sterol in the lipid mixture is cholesterol.

In some embodiments, the population of platelets is transfected with an LNP composition comprising a mRNA encoding the exogenous protein and a lipid mixture, wherein the at least one polyethylene glycol (PEG)-lipid conjugate in the lipid mixture is 1,2-dimyristoyl-rac-glycero-3-methoxypolyethylene glycol-2000 (DMG-PEG2000), 1,2-distearoyl-sn-glycero-3-phosphoethanolamine-N-amino (polyethylene glycol)-2000 (DSPE-PEG2000), or PEG-1,2-distearoyl-rac-glycero-3-methylpolyoxyethylene 2000 (DSG-PEG). In other embodiments, population of platelets is transfected with an LNP composition comprising a mRNA encoding the exogenous protein and a lipid mixture, wherein the at least one polyethylene glycol (PEG)-lipid conjugate in the lipid mixture is 1,2-dimyristoyl-rac-glycero-3-methoxypolyethylene glycol-2000 (DMG-PEG2000).

In some embodiments, a population of platelet cells is transfected with an LNP composition comprising a mRNA encoding the exogenous protein and a lipid mixture, wherein the at least one ionizable cationic lipid in the lipid mixture is CL4H6 and the helper lipid in the lipid mixture is phosphatidylcholine (POPC). In other embodiments, the population of platelet cells is transfected with an LNP composition comprising a mRNA encoding the exogenous protein and a lipid mixture, wherein the at least one ionizable cationic lipid in the lipid mixture is ALC-0315 and the helper lipid is 1,2-dioleoyl-sn-glycero-3-phosphocholine (DOPC).

In some embodiments, the population of platelet cells is transfected with an LNP composition comprising a mRNA encoding the exogenous protein and a lipid mixture, wherein the messenger RNA present in the LNP composition comprises one or more nucleotide modifications, such as 5-methoxyuridine, pseudouridine, or N¹-methylpseudouridine.

### BRIEF DESCRIPTION OF THE DRAWINGS

FIG. 1A shows a schematic of the mRNA-LNP transfection process for donor platelets.

FIG. 1B shows exogenous reporter protein expression in platelets that are either untreated, or transfected with mRNA encoding reporter protein luciferase delivered without agents (‘Naked mRNA’), complexed with Lipofectamine, complexed with RiboJuice, or encapsulated in mRNA-LNP (‘Optimized’).

FIG. 1C shows mRNA uptake by platelets that are either untreated (no mRNA), or transfected with Cy5-labeled mRNA delivered without agents (‘Naked mRNA’), complexed with Lipofectamine, complexed with RiboJuice, or encapsulated in mRNA-LNP (‘Optimized’).

FIG. 1D shows activation levels and percentage of CD62P—positive platelets that are either untreated, or transfected with mRNA encoding reporter protein luciferase delivered without agents (‘Naked mRNA’), complexed with Lipofectamine, complexed with RiboJuice, or encapsulated in mRNA-LNP (‘Optimized’).

FIG. 2A shows exogenous protein expression in platelets transfected with mRNA-LNP formulated with varied ionizable lipid components.

FIG. 2B shows activation levels and percentage of CD62P—positive cells in platelets transfected with mRNA-LNP formulated with varied ionizable lipid components.

FIG. 2C shows exogenous protein expression in platelets transfected with mRNA-LNP formulations comprising varied ionizable and structural (helper) lipid components.

FIG. 2D shows activation levels and percentage of CD62P—positive cells in platelets transfected with mRNA-LNP formulations comprising varied ionizable and structural (helper) lipid components.

FIG. 2E shows exogenous protein expression by mRNA-LNP in the MEG-01 cell line.

FIG. 2F shows exogenous protein expression in apheresis platelets transfected with mRNA-LNP.

FIG. 2G shows exogenous protein expression in platelets transfected with pseudouridine—modified mRNA encoding firefly luciferase, encapsulated in LNP comprised of lipid components CL4H6 and POPC.

FIG. 3A shows platelet responsiveness to agonists ADP and thrombin, following transfection with LNP comprised of CL4H6 and POPC.

FIG. 3B is a rotational thromboelastometry (ROTEM) trace showing the range of clot firmness over time of whole blood (WB), or platelets treated with LNP ALC-0315 DOPC or CL4H6 POPC combined with plasma and red blood cells (TP), spiked into diluted whole blood (DWB).

FIG. 3C shows clot formation time of control or LNP-transfected platelets in rotational thromboelastometry after activation by thromboplastin.

FIG. 3D shows maximum clot firmness of control or LNP-transfected platelets in rotational thromboelastometry after activation by thromboplastin.

FIG. 3E shows clot formation time of control or LNP-transfected platelets in rotational thromboelastometry after activation by ellagic acid.

FIG. 3F shows maximum clot firmness of control or LNP-transfected platelets in rotational thromboelastometry after activation by ellagic acid.

FIG. 4A is a Pearson correlation matrix between exogenous protein expression, mRNA uptake, and platelet activation in platelets transfected with mRNA-LNP.

FIG. 4B is a correlation plot between exogenous protein expression and mRNA uptake in platelets transfected with mRNA-LNP.

FIG. 4C is a correlation plot between exogenous protein expression and platelet activation in platelets transfected with mRNA-LNP.

FIG. 4D shows intracellular exogenous protein in platelets transfected with mRNA-LNP, following activation with agonists ADP or thrombin.

FIG. 4E shows extracellular exogenous protein in platelets transfected with mRNA-LNP, following activation with agonists ADP or thrombin.

FIG. 5A shows exogenous protein synthesis in platelets either untransfected (No mRNA) or transfected with mRNA-LNP, incubated with or without cycloheximide (CHX).

FIG. 5B shows mRNA uptake in platelets without transfection (No mRNA) or transfected with mRNA-LNP, incubated with or without cycloheximide (CHX). mRNA uptake shown as flow cytometry histograms (left image) and quantified mean fluorescence intensity (right image).

FIG. 6A shows mRNA uptake (left axis, bars) and percent transfected cells (right axis, circles) in platelets transfected with mRNA-LNP formulated with varied ionizable lipid components.

FIG. 6B shows mRNA uptake (left axis, bars) and percent transfected cells (right axis, circles) in platelets transfected with mRNA-LNP formulations comprising varied ionizable and structural (helper) lipid components.

FIG. 7A shows exogenous protein expression (left axis, bars) in platelets transfected with mRNA-LNP formulated with various molar percentages of PEG-lipid conjugate. LNP particle diameter (right axis) is shown in grey circles.

FIG. 7B shows mRNA uptake (left axis, bars) and percentage of transfected cells (right axis, circles) in platelets transfected with mRNA-LNP formulated with various molar percentages of PEG-lipid conjugate.

FIG. 7C shows activation levels (left axis, bars) and percentage of CD62P—positive cells (right axis, circles) in platelets transfected with mRNA-LNP formulated with various molar percentages of PEG-lipid conjugate.

FIG. 8A shows exogenous protein expression in platelets transfected with LNP encapsulating nanoluciferase-encoding mRNA with indicated uridine base analogs.

FIG. 8B shows activation levels and percentage of CD62P—positive cells in platelets transfected with LNP encapsulating nanoluciferase-encoding mRNA with indicated uridine base analogs.

FIG. 8C shows exogenous protein expression in platelets transfected with LNP encapsulating nanoluciferase-encoding mRNA with indicated uridine base analogs.

FIG. 9A shows prothrombin time in rat whole blood before and after polytrauma.

FIG. 9B shows exogenous protein in rat donor platelets before (Pre-Transfusion) and after (Post-Transfusion) circulation in recipient animal models of polytrauma.

FIG. 9C shows kidney bleeding time of rats before (Baseline) and after (Post-Trauma) polytrauma and resuscitation with either normal, or mRNA-LNP transfected, rat platelets.

FIG. 9D shows blood loss from kidney bleeding assay in rats before (Baseline) and after (Post-Trauma) polytrauma and resuscitation with either normal, or mRNA-LNP transfected, rat platelets.

### DETAILED DESCRIPTION

The present invention discloses novel lipid nanoparticle compositions that are internalized by platelets, ex vivo, with supported intracellular mRNA release once inside, and downstream expression of the encoded protein.

### Definitions

“Activation” or “activated”, as used herein in the context of platelets, refers to ability of platelets to aggregate and promote blood clotting, as measured by any method or assay known to one of skill in the art. For example, activation of platelets may be measured by monitoring levels of cell surface exposure of CD62P, a marker of platelet activation, by flow cytometry.

“Cationic lipid”, as used herein, a protonatable tertiary amine (e.g., pH titratable) head group, C16 to C18 alkyl chains, ether linkages between the head group and alkyl chains, and 0 to 3 double bonds.

“Ionizable cationic lipid” or “ICL”, as used herein, is a cationic lipid that is protonated and positively charged at low pH but remains neutral at physiological pH.

“Exogenous”, as used herein, refers to a messenger RNA (mRNA) or protein that is not naturally found in, or produced by, platelet cells.

“Helper lipid”, as used herein, refers to any vesicle-forming lipid (e.g., bilayer-forming lipid) lipid, other than the ICL, that is added to a lipid nanoparticle to enable or enhance the ability of the mRNA within the LNP to transfect a platelet cell.

“Lipid nanoparticle(s)” or “LNP(s)”, as used herein, are spherical vesicles made of one or more ionizable cationic lipid, one or more helper lipid, cholesterol (to fill the gaps between the lipids), and one more polyethylene glycol (PEG)-lipid conjugate, plus the nucleic acid (DNA or RNA) cargo within the particle.

“Minimal activation of platelets” or “minimally activated”, as used herein, refers to maintaining a state of platelet activation which is less than the level seen in platelets treated with agonist thrombin, following preparation for transfection.

“Mole percent” or “mol %”, as used herein, is the percentage of the moles of a particular component relative to total moles of all components that are in a mixture.

“Polyethylene glycol-lipid conjugate” or “PEG-lipid” or “PEGylated lipids”, as used herein, refers to derivatives of polyethylene glycol (PEG) covalently attached to a lipid moiety;

### Lipid Nanoparticle (LNP) Compositions

The present invention relates to novel LNP compositions useful for transfection of platelet cells with exogenous messenger RNA (mRNA-LNP) and expression of the encoded endogenous protein in the resulting transfected platelet cells.

Lipid nanoparticles of the present invention comprise a mRNA encoding an exogenous protein and a lipid mixture comprising an ionizable cationic lipid (ICL), a helper lipid, a sterol, and a polyethylene glycol (PEG)-lipid conjugate. The inventors have found novel lipid mixtures of specific ICLs and helper lipids which, when combined with a sterol and a PEG-lipid conjugate, enable the expression of exogenous proteins in transfected platelet cells. These specific ICLs and helper lipids are shown in Table 1:

TABLE 1
Ionizable Cationic Lipids (ICLs) and Helper Lipids
for Transfection of Platelet Cells with LNP

| ICL | Helper Lipid |
|---|---|
| CL1D6^{a} | phosphatidylcholine (POPC) |
| CL1H6 ^{a} | 1,2-dioleoyl-sn-glycero-3-phosphocholine (DOPC) |
| CL4H6 ^{a} | 1,2-distearoyl-sn-glycero-3-phosphocholine (DSPC) |
| CL15H6 ^{a} | 1,2-dioleoyl-sn-glycero-3-phosphoethanolamine (DOPE) |
| SM-102 ^{b} | dioleoyl phosphatidylglycerol (DOPG) |
| ALC-0159^{c} | egg sphingomyelin (ESM) |
| ALC-0315^{c} | |

^{a} US2020/0129431A1
^{b} Hassett, K. J. et al. Optimization of lipid nanoparticles for intramuscular administration of mRNA vaccines. Mol. Ther. Nucleic Acids 15, 1-11 (2019).
^{c}Ansell S M, Du X. Lipids and lipid nanoparticle formulations for delivery of nucleic acids. U.S. Pat. No. 10,166,298B2. 2019.

### Lipid Mixtures

The LNPs of the present invention may include one or more of the following ICLs in the lipid mixture: CL4H6, SM-102, ALC-0315, CL1H6, CL15H6, CL1D6, ALC-0159 or any combination thereof. For example, the lipid mixture may include CL4H6 or ALC-0315 as the ICL.

The lipid mixture in the LNPs of the present invention may include one or more of the following helper lipids: phosphatidylcholine (POPC), 1,2-dioleoyl-sn-glycero-3-phosphocholine (DOPC), 1,2-distearoyl-sn-glycero-3-phosphocholine (DSPC), 1,2-dioleoyl-sn-glycero-3-phosphoethanolamine (DOPE), dioleoyl phosphatidylglycerol (DOPG), egg sphingomyelin (ESM), or any combination thereof. For example, the lipid mixture may include phosphatidylcholine (POPC) or 1,2-dioleoyl-sn-glycero-3-phosphocholine (DOPC) as the helper lipid.

The LNPs of the present invention may include one or more of the following sterols in the lipid mixture: cholesterol or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol, or any mixture thereof. For example, the LNPs of the present invention may include cholesterol in the lipid mixture.

The LNPs of the present invention may include one or more of the following polyethylene glycol (PEG)-lipid conjugate in the lipid mixture: 1,2-dimyristoyl-rac-glycero-3-methoxypolyethylene glycol-2000 (DMG-PEG2000), 1,2-distearoyl-sn-glycero-3-phosphoethanolamine-N-amino(polyethylene glycol)-2000 (DSPE-PEG2000), or PEG-1,2-distearoyl-rac-glycero-3-methylpolyoxyethylene 2000 (DSG-PEG). For example, the LNPs of the present invention may include 1,2-dimyristoyl-rac-glycero-3-methoxypolyethylene glycol-2000 (DMG-PEG2000) in the lipid mixture.

Suitable lipid mixtures for use in the LNPs of the present invention may comprise, for example, CL4H6 as the ICL in combination with phosphatidylcholine (POPC) as the helper lipid. Other suitable lipid mixtures for use in the LNPs of the present invention may comprise, for example, ALC-0315 as the ICL and 1,2-dioleoyl-sn-glycero-3-phosphocholine (DOPC) as the helper lipid.

In the lipid mixture of the LNP of the present invention, the ICL, helper lipid, sterol and PEG-lipid conjugate are mixed to achieve a desired mole percent of each component. For example, the mole percent of the ICL may range from 30% to 55%, or any mole percent therebetween, for example the mole percent of the ICL may be 30%, 35%, 40%, 45%, 46%, 47%, 48%, 49%, 50%, 51%, 52%, 53%, 54%, 55%, or any value therebetween; the mole percent of the helper lipid may range from 5% to 20%, or any mole percent therebetween, for example, the mole percent of the helper lipid may be 5%, 6%, 7%, 8%, 9%, 10%, 11%, 12%, or any value therebetween; the mole percent of the sterol may range from 25% to 50%, or any mole percent therebetween, for example, the mole percent of the sterol may be 25%, 27.5%, 30%, 32.5%, 35%, 36%, 37%, 38%, 38.5%, 39%, 39.5%, 40%, 42%, 44%, 46%, 48%, 50%, or any value therebetween; and the mole percent of polyethylene glycol (PEG)-lipid conjugate may range from 0.5% to 3%, or any mole percent therebetween, for example, the mole percent of polyethylene glycol (PEG)-lipid conjugate may be 0.5%, 1%, 1.5%, 2%, 2.5%, 3%, or any value therebetween. For example, the mole percent of the ICL be 50%; the mole percent of the helper lipid may be 10%; the mole percent of the sterol may be 38.5%; and the mole percent of polyethylene glycol (PEG)-lipid conjugate may be 1.5%.

In some embodiments, the lipid mixture in the LNP particles comprises 50 mole percent CL4H6, 10 mol % phosphatidylcholine (POPC), 38.5% cholesterol, and 1.5% 1,2-dimyristoyl-rac-glycero-3-methoxypolyethylene glycol-2000 (DMG-PEG2000). In other embodiments, the lipid mixture in the LNP particles comprises 50 mole percent ALC-0315, 10 mol % 1,2-dioleoyl-sn-glycero-3-phosphocholine (DOPC), 38.5% cholesterol, and 1.5% 1,2-dimyristoyl-rac-glycero-3-methoxypolyethylene glycol-2000 (DMG-PEG2000.

### Messenger RNA

The LNPs of the present invention, in addition to the above-described lipid mixture, also comprise messenger RNA (mRNA). The messenger RNA may encode an exogenous protein that is not inherent to or produced by unmodified platelet cells. Examples of exogenous proteins that may be encoded by the mRNA contained within the LNP include, but are not limited to: a coagulation factor, an antifibrinolytic protein, an anticoagulant, a fibrinolytic protein, an antimicrobial protein, an immunomodulator, an anti-cancer protein, a genetic editing protein, and a reporter protein. Alternatively, the messenger RNA may not encode an exogenous protein but may be an RNA or DNA aptamer molecule designed or selected to bind to, or otherwise effect the activity of, a specific target biomolecule.

### Use of the LNP Composition

Lipid nanoparticles (LNP) are a flexible and clinically approved platform which enable delivery and intracellular release of genetic material and several methods have been developed in recent years (Novakowski et al, 2019, Scientific Reports 9:552; Sato et al., 2019, J. Controlled Release, 295 (10): 140-152).

The LNP compositions of the present invention, as described above, may be used for transfection of platelet cells with mRNA encoding an exogenous protein to produce a population of platelet cells expressing the exogenous protein. When transfected with the LNP compositions of the present invention, the resulting transfected platelet cells express an exogenous protein encoded by the mRNA.

Transfection of platelet cells with LNP compositions in which the lipid mixture comprises CL4H6 and the ICL and POPC as the helper lipid produce a population of transfected platelet cells which can be subsequently activated in a similar manner and extent as a population of untransfected platelet cells.

### Use of Transfected Platelet Cells Expressing Exogenous Protein

The LNP—treated platelets expressing exogenous protein, or modified proteome, can be applied to specialized or enhanced therapeutic roles for current and future platelet transfusion or treatment indications.

### EXAMPLES

### Materials and Methods

### Blood Component Production

Pooled platelet concentrates (PC), packed red blood cells (pRBCs) and fresh frozen plasma (FFP) were produced by the Canadian Blood Services Blood4Research Facility in Vancouver, Canada, from donors providing signed informed consent. PC was prepared using the buffy coat production method¹¹ and was stored for one day at 22° C. under constant agitation (PC1200-Pro Platelet Incubator, Helmer Scientific, Noblesville, IN). pRBCs were stored at 4° C. and FFP was stored at −30°° C. For some experiments, analogous platelet-poor plasma (PPP) was obtained from platelet units by centrifugation at 2,400×g for 20 min at 22° C. For the model of dilutional coagulopathy, whole blood was collected in citrated Vacutainer tubes (Becton Dickinson, Franklin Lakes, NJ) from healthy donors. This study was conducted in accordance with protocols approved by both the University of British Columbia Ethics Committees (H21-03495 and H16-00773) and the Canadian Blood Services Research Ethics Board (2021-007).

### Platelet Transfection Preparation

Washed platelets (WP) were prepared for transfection from PC units. PC was sampled through a coupling port (Fresenius Kabi, Bad Homburg, Germany) with a sterile Leur-Lok™ syringe (BD, Franklin Lakes, NJ). PC was centrifuged at 250×g for 20 min at room temperature. The supernatant was removed, and the remaining platelet pellet was washed once in anticoagulant citrate dextrose solution, USP, Formula A (ACD-A) (Haemonetics, Boston, MA) and once in Tyrode's-HEPES buffer pH 6.5 (134 mM NaCl, 2.9 mM KCl, 0.34 mM NaH₂PO₄, 10 mM HEPES, 5 mM D-glucose) at 250×g for 10 min at 22° C. WP were suspended in Tyrode's-HEPES buffer pH 6.5 and counted using a Sysmex XN-550 hematology analyzer (Sysmex Corporation, Kobe, Japan). For all experiments, three PC units were washed and transfected within 24 h. Single-donor apheresis platelet units were prepared in the same manner following collection.

### Synthesizing mRNA In Vitro

Messenger RNA was synthesized in bulk by in vitro transcription. Briefly, plasmid DNA templates encoding a CleanCap® AG bacteriophage T7 promoter site and NanoLuc luciferase (NanoLuc) or firefly luciferase coding sequence were linearized with SapI. RNA was produced by in vitro transcription reactions containing CleanCap® AG reagent and modified nucleotides when indicated (TriLink BioTechnologies, San Diego, CA), and purified using a RNeasy Kit (Qiagen, Toronto, ON). RNA was enzymatically tailed using a post-transcriptional tailing kit (CELLSCRIPT, Madison, WI), prior to an additional purification. RNA purity, integrity, and tailing efficiency were monitored by agarose gel electrophoresis. CleanCap® Cyanine-5 Fluc mRNA (5moU) was purchased from TriLink BioTechnologies (San Diego, CA).

### Preparing Lipid Nanoparticles Containing mRNA

An ethanolic lipid mixture containing an ionizable lipid, helper lipid, cholesterol, and 1,2-dimyristoyl-rac-glycero-3-methoxypolyethylene glycol-2000 (DMG-PEG2000) (50:10:38.5:1.5 mol %) was mixed via a T-junction mixer at a 1:3 ratio with an aqueous solution of 25 mM sodium acetate pH 4 containing the desired mRNA at an amine-to-phosphate (N/P) ratio of 6. The resulting mixture was dialyzed (Spectra/Por 2 Dialysis Tubing 12-14 kDa MWCO, Spectrum Labs, San Francisco, CA) 500-fold against 1× phosphate-buffered saline (PBS) and then sterile-filtered through an Acrodisc® 0.2 μm syringe filter (Pall Corporation, Mississauga, ON). The formulations were concentrated in Amicon® 10,000 kDa MWCO ultracentrifugation units (EMD Millipore Corporation, Billerica, MA) and the RNA content and encapsulation determined using a Quant-it™ Ribogreen RNA Assay Kit (Thermo Fisher Scientific, Eugene, OR). Total lipid content was determined with the Cholesterol E kit (Fujifilm Wako Diagnostics, Mountain View, CA). Particle size and polydispersity index were measured via dynamic light scattering on the Malvern Zetasizer Nano (Malvern Panalytical, Worcestershire, England).

### Treating Platelets With mRNA-LNP

Washed platelets were treated with mRNA-LNP at a ratio of 12 μg mRNA per 40×10⁶ platelets at a concentration of 40×10⁶ platelets mL⁻¹ in Tyrode's-HEPES buffer pH 6.5 and incubated at 37° C. for 4 h before further downstream processing. When treating with the commercial reagents, Lipofectamine™ MessengerMAX™ (Thermo Fisher Scientific, Carlsbad, CA) and RiboJuice™ (EMD Millipore Corporation, Burlington, MA), the platelets were treated with equivalent amounts of mRNA and according to the manufacturer's recommended protocols. Following transfection, exogenous protein expression (by enzymatic assay of NanoLuc activity), mRNA uptake (by Cyanine 5-conjugated mRNA), and platelet activation (by cell-surface expression of CD62P) were assessed.

### Luciferase Assay

Washed and treated platelets were pelleted by centrifugation at 300×g for 10 min at room temperature before lysing in 100 μL of Glo Lysis buffer (Promega Corporation, Madison, WI). Lysates were transferred to a 96-Flat Well white microplate and mixed with prepared NanoLuc substrate (Promega Corporation, Madison, WI) at a 1:1 ratio. Luminescence was recorded on the Tecan Spark® Multimode Microplate Reader (Paramit Corporation, Morgan Hill, CA) with an integration time of 1000 ms and zero attenuation. The total protein content in each lysate was determined using the Pierce™ BCA Protein Assay Kit (Thermo Fisher Scientific, Rockford, IL) according to the manufacturer's protocol, with absorbance measured at 562 nm on the Tecan Spark Multimode Microplate Reader. NanoLuc expression was then reported as the luminescence normalized to the total protein content.

### Flow Cytometry

Washed and treated platelets were incubated in Tyrode's-HEPES buffer pH 6.5 at 20×10⁶ platelets mL⁻¹ with FITC-conjugated mouse anti-human CD42b (HIP1, 11-0429-42, Invitrogen) and PE-conjugated mouse anti-human CD62P (AC1.2, 550561, BD Pharmingen) antibodies for 30 min at room temperature. FITC-conjugated mouse IgG1 κ (P3.6.2.8.1, 11-4714-82, Invitrogen) and PE-conjugated mouse IgG1 κ (MOPC-21, 556650, BD Pharmingen) were used as isotype controls. All antibodies were diluted at a ratio of 1:25. Samples were further diluted ten-fold in Tyrode's-HEPES buffer pH 6.5 following incubation and analyzed on the CytoFLEX LX Flow Cytometer (Beckman Coulter, Indianapolis, IN), using a consistent gating strategy with approximately 25,000 total events collected. All flow cytometry data was analyzed using FlowJo v10.8.1.

### Characterizing LNP Treated Platelets by Rotational Thromboelastometry

The effect of LNP treatment on the hemostatic profile of transfected platelets was assessed using Rotational Thromboelastometry (ROTEM) and a model of in vitro trauma transfusion. Diluted whole blood (DWB) was combined with a transfusion package (TP) consisting of blood products combined at a ratio of 1 pRBC unit: 1 plasma unit: 1 platelet unit prior to addition into the ROTEM cup. Whole blood (WB) was diluted to 20% hematocrit (L/L) using 0.9% (w/v) saline solution, pH 5.5 (Baxter Corporation, Deerfield, IL) to simulate dilutional coagulopathy prior to adding the TP. Platelet components used to make the TP include washed platelets that were untreated, or treated with ALC-0315 DOPC or CL4H6 POPC LNP not containing mRNA. LNP were dosed at 500 μg lipid per 40 million platelets, an equivalent lipid dosage to the mRNA-LNP used in composition screens. Untreated and LNP-treated platelets were normalized to a concentration of 300×10⁶ platelets mL⁻¹ using Tyrode's-HEPES buffer pH 6.5. Once prepared TPs were spiked into hemodiluted WB at a ratio of 70% transfusion package: 30% hemodiluted WB, to model transfusion of the TP into coagulopathic patients with hemodiluted WB. The hemostatic profile was measured by ROTEM delta (Werfen, Bedford, MA). Samples comprised of either WB, hemodiluted WB, or hemodiluted whole blood mixed with TPs, and were added to the ROTEM cup to a final volume of 300 μL. Both intrinsic pathway (INTEM) and extrinsic pathway (EXTEM) assays were performed for three biological replicates. STAR-TEM and INTEM or EXTEM reagents, at a volume of 20 μL each, were added automatically by the ROTEM machine. Reactions were allowed to run for 30 min. Key parameters of the ROTEM profile reported include clot formation time (CFT), maximum clot firmness (MCF), alpha angle (measure of clotting kinetics), clot firmness at 10 (A10) and 20 (A20) minutes.

### Evaluating Platelet Responsiveness to Agonists

Platelets untreated or treated with CL4H6 POPC LNP were activated after 2 h with 0.1 U mL-1 thrombin from human plasma (MilliporeSigma Canada Ltd, Oakville, ON) or 10 μM Chrono-Par® ADP reagent (Chrono-Log, Havertown, PA) in Tyrode's-HEPES buffer pH 7.4 (134 mM NaCl, 2.9 mM KCl, 0.34 mM NaH₂PO₄, 10 mM HEPES, 5 mM D-glucose). After 4 h LNP treatment, platelets were collected for flow cytometry and the remaining sample was centrifuged at 250×g for 10 min, with the pellet and supernatant fractions collected for luciferase assay.

### Statistical Analysis

All experiments consist of three biological replicates, with all values expressed as mean ± the standard error of the mean. Analyses between two groups was performed using either a one-way unpaired Student or Welch's t-test, while grouped analyses were performed using a one-way analysis of variance (ANOVA) followed by a Bonferroni's post-hoc correction. Assumptions on variance were determined using an F-test or Brown-Forsythe test as appropriate, while normality was assessed via a Shapiro-Wilks test. Statistical analysis and graphs were generated using GraphPad Prism v9.0. A P-value<0.05, 95% confidence intervals, was considered significant.

### Example 1: Platelets Transfected With mRNA-LNP Express Exogenous Protein

This example demonstrates that LNP enable uptake and translation of the delivered mRNA by platelets ex vivo.

Enzymatic assay of reporter protein nanoluciferase (NanoLuc) activity was used to measure exogenous protein levels in human platelets transfected using mRNA either uncomplexed, delivered via commercial reagents, or encapsulated in LNP, as described in the Materials and Methods.

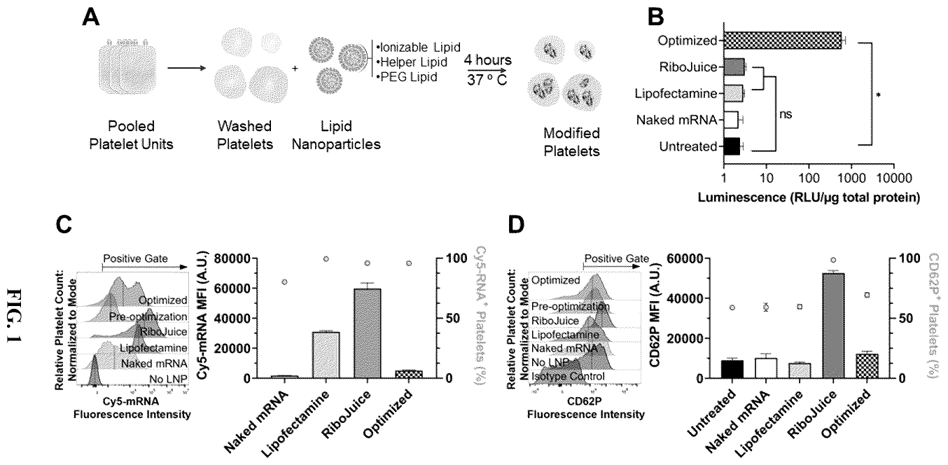

The results are shown in FIG. 1B. As can be seen in FIG. 1B, exogenous protein synthesis is only evident in platelets transfected with mRNA encapsulated in LNP, with other mRNA delivery systems yielding no detectable levels exogenous protein above untreated cells.

mRNA uptake and platelet activation during transfection were monitored by flow cytometry as described in the Materials and Methods, using Cyanine-5 (Cy5) conjugated mRNA and PE-conjugated mouse anti-human CD62P labeling, respectively.

Together with FIG. 1B, FIG. 1C shows that while commercial reagents support mRNA uptake by platelets, they do not induce translation of delivered mRNA, and thus synthesis of the exogenous protein.

As seen in FIG. 1D, transfection with LNP also limits the activation state of platelets, maintaining a level amenable to therapeutic use. FIG. 1D also shows that commercial reagents induce minor to considerable levels of platelet activation during mRNA delivery.

### Example 2: Platelet-Specific Lipid Composition of mRNA-LNP

This example demonstrates that platelet transfection requires specialized LNP components, including ionizable lipids and RNA base modifications.

Enzymatic activity assays activity were used to measure exogenous protein levels in human platelets transfected with mRNA encoding nanoluciferase (NanoLuc) encapsulated in LNP formulated with varied ionizable lipid components, as described in the Materials and Methods.

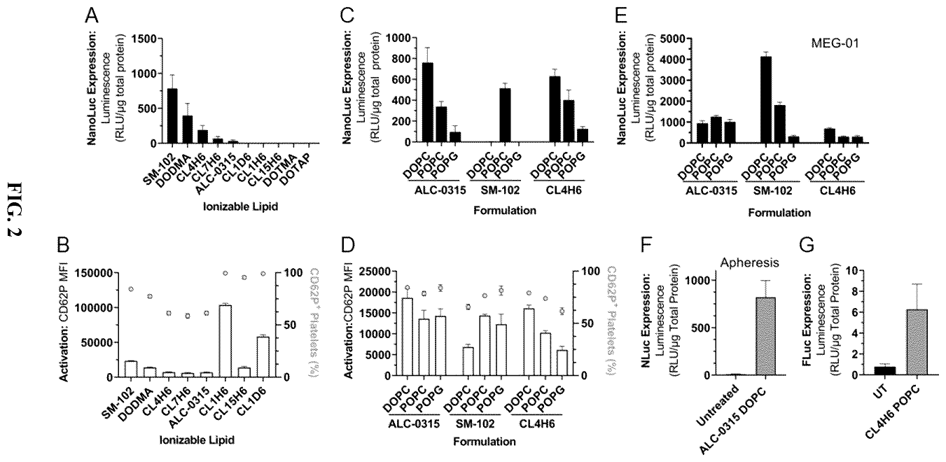

The results are shown in FIG. 2A. As seen in FIG. 2A, the ionizable lipid component has a significant effect on mRNA translation and protein expression in LNP-mediated platelet transfection.

Platelet activation during transfection was monitored by flow cytometry as described in the Materials and Methods, using PE-conjugated mouse anti-human CD62P labeling.

As shown in shown in FIG. 2B, the ionizable lipid component has a significant effect on both the levels of platelet activation, and the percentage of platelets which are activated during transfection.

Enzymatic activity assays were used to measure exogenous protein levels in human platelets transfected with mRNA encoding nanoluciferase (NanoLuc) encapsulated in LNP formulations comprising varied ionizable and structural (helper) lipid components, as described in the Materials and Methods. Platelet activation during transfection was monitored by flow cytometry as described in the Materials and Methods, using PE-conjugated mouse anti-human CD62P labeling.

As shown in FIG. 2C, a combinatorial approach elucidated further LNP formulations capable of inducing exogenous protein expression in platelets. Formulations containing ALC-0315 DOPC, CL4H6 POPC, CL4H6 DOPC, and SM102 POPC supported the highest levels of protein expression.

The results of platelet activation within this LNP screen are shown in FIG. 2D. As seen in FIG. 2D, combinations of ionizable and structural (helper) lipids yield varying degrees of platelet activation during transfection.

Enzymatic activity assays were used to measure exogenous protein levels in MEG-01 cells transfected with mRNA encoding nanoluciferase (NanoLuc) encapsulated in LNP formulations comprising varied ionizable and structural (helper) lipid components, as described in the Materials and Methods.

FIG. 2E shows the expression levels of nanoluciferase in MEG-01 cells. As can be seen in FIG. 2E and FIG. 2C, there is little correlation of LNP efficacy in platelet cells versus MEG-01 cells, as measured by nanoluciferase expression.

Enzymatic activity assays were used to measure exogenous protein levels in human platelets prepared from single donors by apheresis, transfected with mRNA encoding nanoluciferase (NanoLuc) encapsulated in LNP formulation using ALC0315 and DOPC, as described in the Materials and Methods.

The results are shown in FIG. 2F. As seen in FIG. 2F, platelets prepared by apheresis and transfected with mRNA-LNP are able to produce functional exogenous protein.

Enzymatic activity assays were used to measure exogenous protein levels in platelets transfected with pseudouridylated mRNA encoding fireflyluciferase (FLuc), encapsulated in LNP formulation CL4H6 POPC, as described in the Materials and Methods.

As seen in FIG. 2G, transfected platelets expressed functional FLuc luciferase, a protein unrelated to nanoluciferase.

### Example 3: In Vitro Functionality of mRNA-LNP Transfected Platelets

This example shows that LNP-transfected platelets retain agonist responsiveness and clotting function in vitro.

Platelet responsiveness to agonists ADP or thrombin was measured by flow cytometry staining using PE-conjugated mouse anti-human CD62P labeling, as described in the Materials and Methods.

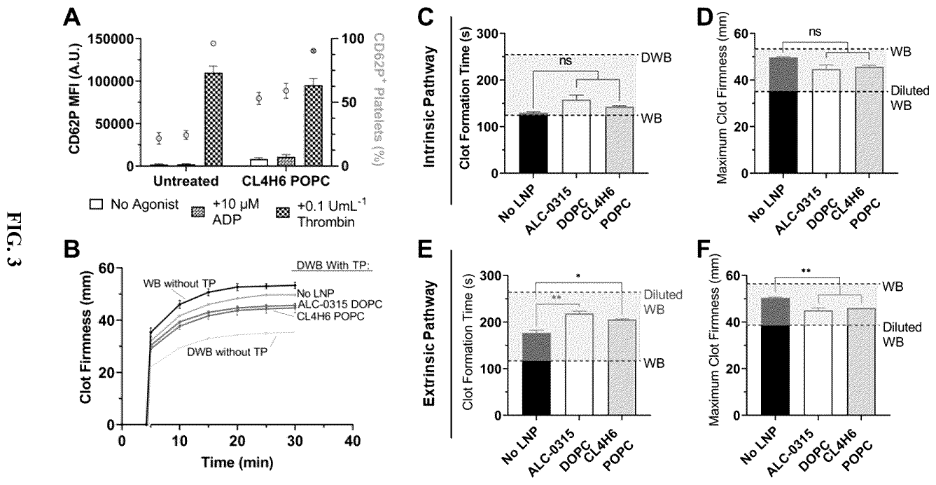

The results are shown in FIG. 3A. As seen in FIG. 3A, platelets transfected with CL4H6 POPC LNP maintained responsiveness to platelet agonists. Transfected platelets responded similarly to control untreated platelets, where ADP and thrombin mildly and substantially increased CD62P levels, respectively.

Rotational thromboelastometry (ROTEM) as described in the Materials and Methods was used to measure clotting properties in vitro of LNP-transfected platelets.

The results are shown in FIG. 3B, FIG. 3C, FIG. 3D, FIG. 3E, and FIG. 3F. As seen in the ROTEM trace in FIG. 3B, platelets transfected with LNP ALC-0315 DOPC or CL4H6 POPC were coagulable in a manner similar to untreated platelets, in whole blood.

FIG. 3C and FIG. 3D compare clot formation time and maximum clot firmness in whole blood with untreated (No LNP) platelets, or platelets transfected with LNP ALC-0315 DOPC or CL4H6 POPC, with clotting initiated through the intrinsic pathway. As seen in FIG. 3C, there was no statistically significant difference in clot formation time between control platelets and LNP-treated platelets. As shown in FIG. 3D, there was no statistically significant difference in the maximum clot firmness between control platelets and LNP-treated platelets.

FIG. 3E and FIG. 3F compare clot formation time and maximum clot firmness in whole blood with untreated (No LNP) platelets, or platelets transfected with LNP ALC-0315 DOPC or CL4H6 POPC, with clotting initiated through the extrinsic pathway. As shown in FIG. 3E, clot formation time was higher in LNP-treated platelet than untreated platelets, but lower than diluted whole blood alone. As shown in FIG. 3D, maximum clot firmness of LNP-treated platelet samples was decreased compared to untreated platelet samples, but higher than in diluted whole blood alone.

### Example 4: Expression of Exogenous Protein Does Not Correlate With Activation Nor Extent of mRNA Uptake

This example shows that exogenous protein expression in transfected platelets neither correlates nor requires platelet activation, and does not correlate with mRNA-LNP uptake.

Correlation between NanoLuc expression and mRNA uptake and platelet activation was performed, and a correlation matrix was created, as described in the Materials and Methods.

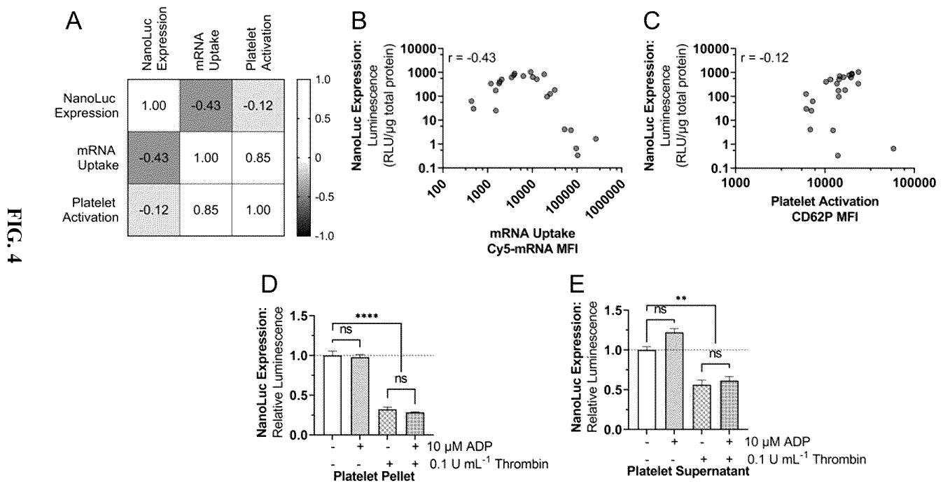

The results are shown in FIG. 4A, FIG. 4B, and FIG. 4C. As shown in FIG. 4A-C, expression of exogenous NanoLuc did not correlate strongly with either mRNA uptake or platelet activation.

Platelets were transfected with mRNA-LNP encoding NanoLuc, followed by activation with agonists ADP or thrombin. Intracellular and extracellular protein was separated by centrifugation, and enzymatic activity assays were used to measure exogenous protein levels in both fractions as described in the Materials and Methods.

As seen in FIG. 4D and FIG. 4E, both intracellular and extracellular NanoLuc levels were unchanged when transfected platelets were stimulated by ADP. Stimulation by thrombin decreased NanoLuc protein expression in both cell pellet and supernatant fractions.

### Example 5: Protein Synthesis Inhibition Does Not Impede LNP Uptake

This example shows that an inhibitor of global protein synthesis can prevent target production, without impacting mRNA-LNP uptake.

Platelets were transfected with mRNA encoding nanoluciferase (NanoLuc) encapsulated in LNP formulation using ALC0315 and DOPC immediately preceding treatment with cycloheximide, followed by enzymatic assays of nanoluciferase activity, and activation by flow cytometry using PE-conjugated mouse anti-human CD62P labeling, as described in the Materials and Methods.

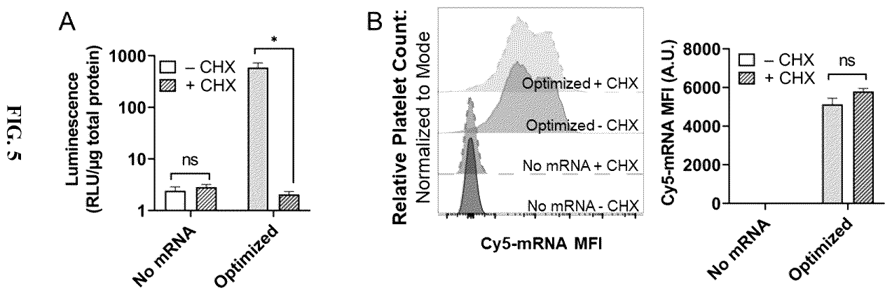

The results are shown in FIG. 5A and FIG. 5B. As seen in FIG. 5A, exogenous protein synthesis in transfected platelets was inhibited by cycloheximide treatment. As seen in FIG. 5B, the presence or absence of cycloheximide had no statistically significant effect on mRNA-LNP uptake in transfected platelets.

### Example 6: Lipid Type Impacts LNP Uptake by Platelets

This example shows that the lipid composition of LNP impacts mRNA uptake by platelets.

mRNA uptake by platelets transfected with LNP of varied lipid composition was monitored by flow cytometry as described in the Materials and Methods, using Cy-5 labeled mRNA.

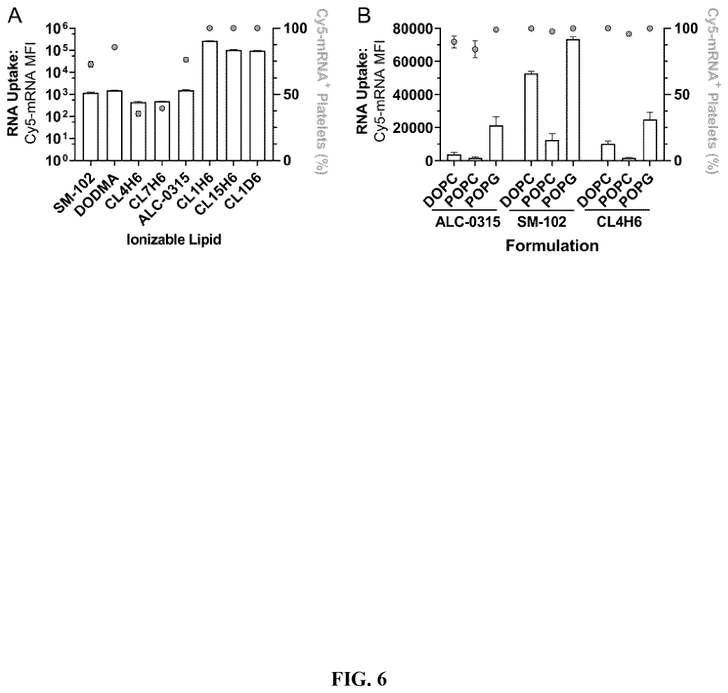

The results are shown in FIG. 6A and FIG. 6B. As shown in FIG. 6A, the ionizable lipid significantly influences both the extent of mRNA uptake by platelets, and the percentage of platelets which take up mRNA-LNP. As shown in FIG. 6B, the combination of ionizable and structural (helper) lipid influences both the extent of mRNA uptake by platelets, and the percentage of platelets which take up mRNA-LNP.

### Example 7: PEG Composition of LNP Affects mRNA Uptake and Platelet Activation

This example shows that the molar percentage of PEG in LNP formulations affects the mRNA uptake and activation of transfected platelets.

Enzymatic activity assays were used to measure exogenous protein levels in platelets transfected with mRNA-LNP encoding nanoluciferase (NanoLuc), formulated with varied molar percentage of PEG lipid, as described in the Materials and Methods.

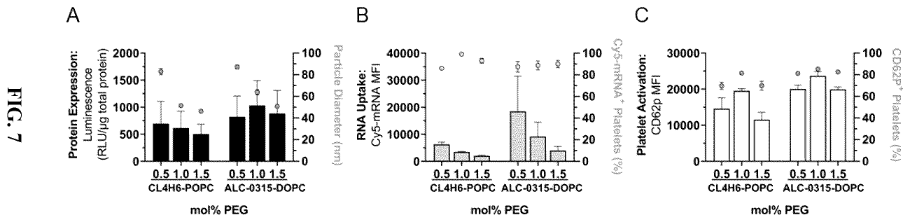

The results are shown in FIG. 7A. As can be seen in FIG. 7A, the molar percentage of PEG has a limited effect on exogenous protein expression in platelet transfection.

mRNA uptake and platelet activation during transfection were monitored by flow cytometry as described in the Materials and Methods, using Cyanine-5 (Cy5) conjugated mRNA and PE-conjugated mouse anti-human CD62P labeling, respectively.

The results are shown in FIG. 7B and FIG. 7C. As seen in FIG. 7B, the molar percentage of PEG influences the levels of mRNA uptake, and the percentage of LNP-transfected platelets. As seen in FIG. 7C, the molar percentage of PEG in LNP formulations can significantly influence the extent of platelet activation, and the percentage of platelets that are activated.

### Example 8: mRNA Modifications for Platelet-Specific Enhancement of Expression

This example shows that certain RNA modifications can enhance expression in platelets transfected with mRNA-LNP.

mRNA was prepared using various uridine analogs prior to encapsulation by LNP and administration to platelets, as described in the Materials and Methods. Enzymatic activity assays were used to measure exogenous protein levels in transfected platelets, and platelet activation was monitored by flow cytometry using PE-conjugated mouse anti-human CD62P labeling, as described in the Materials and Methods.

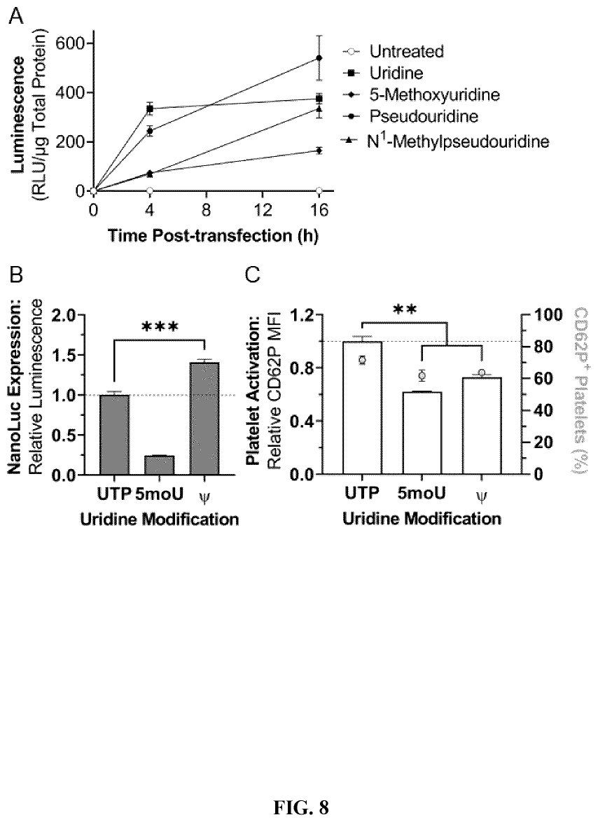

The results are shown in FIG. 8A and FIG. 8B. As shown in FIG. 8A, pseudouridylation of the delivered mRNA enhanced protein expression at later time points. Incorporation of either 5-methoxyuridine or N¹-methylpseudouridine modified the kinetics of proteins expression in platelets. As seen in FIG. 8B, exogenous protein expression at 4 hours is significantly improved when mRNA is pseudouridylated. Incorporation of 5-methoxyuridine in the mRNA significantly decreased exogenous protein expression.

As seen in FIG. 8C, both 5-methoxyuridine and pseudouridine modifications significantly decreased CD62P levels of transfected platelets, as well as the percentage of activated platelets.

### Example 9: In Vivo Functionality of mRNA-LNP Transfected Platelets

This example shows that mRNA-LNP transfected platelets are capable of safely circulating in vivo, maintaining exogenous protein expression, and participating in hemostasis.

Rat platelets were transfected with mRNA-LNP encoding NanoLuc, and transfused to resuscitate coagulopathic recipient rats following polytrauma and hemorrhage, as described in Materials and Methods.

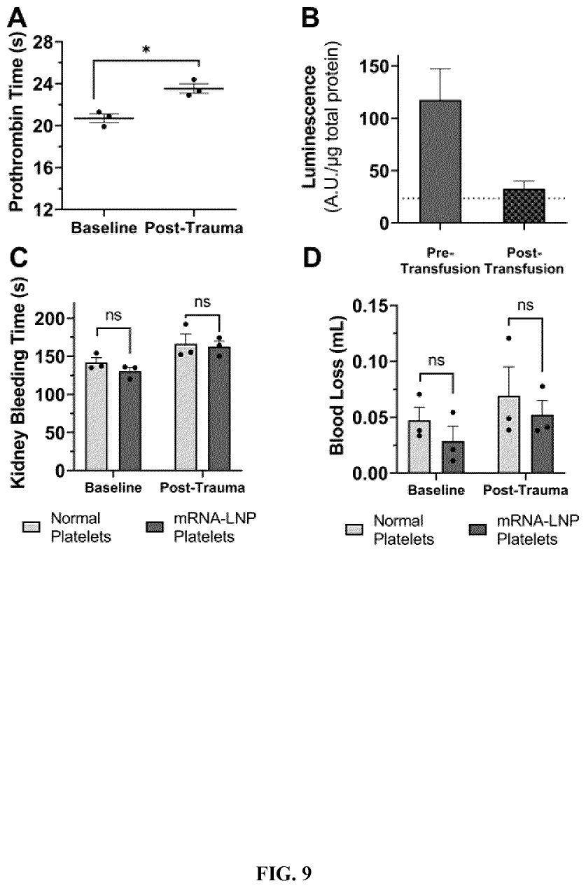

As shown in FIG. 9A, rats receiving resuscitative platelet transfusions had an increase in prothrombin time from baseline to post-trauma time points, confirming a coagulopathic state.

Enzymatic activity assays were used to measure NanoLuc levels in rat platelets transfected with mRNA-LNP both before transfusion (Pre-Transfusion), and after transfusion following isolation from recipient rat whole blood (Post-Transfusion), as described in the Materials and Methods.

As shown in FIG. 9B, exogenous NanoLuc protein was detected in rat blood following in vivo circulation.

Kidney bleeding assays examining platelet transfusion efficacy were carried out on rats both before and after polytrauma and hemorrhage, as described in Materials and Methods.

As shown in FIG. 9C and FIG. 9D, the efficacy of mRNA-LNP transfected platelet transfusion was similar to transfusion of untransfected controls (Normal Platelets). There was no significant difference in kidney bleeding time or volume when rats were treated with normal platelets or mRNA-LNP transfected platelets, following polytrauma.

### REFERENCES

1. Jones J M, Sapiano M R P, Savinkina A A, et al. Slowing decline in blood collection and transfusion in the United States-2017. Transfusion. 2020; 60 Suppl 2: S1-S9.
2. Wang S, Li Z, Xu R. Human Cancer and Platelet Interaction, a Potential Therapeutic Target. Int J Mol Sci. 2018; 19 (4).
3. Johnson J, Wu Y W, Blyth C, Lichtfuss G, Goubran H, Burnouf T. Prospective Therapeutic Applications of Platelet Extracellular Vesicles. Trends Biotechnol. 2021; 39 (6): 598-612.
4. Leung J, Cau M F, Kastrup C J. Emerging gene therapies for enhancing the hemostatic potential of platelets. Transfusion. 2021; 61 Suppl 1: S275-S285.
5. Semple S C, Leone R, Barbosa C J, Tam Y K, Lin P J C. Lipid Nanoparticle Delivery Systems to Enable mRNA-Based Therapeutics. Pharmaceutics. 2022; 14 (2).
6. Weyrich A S, Schwertz H, Kraiss L W, Zimmerman G A. Protein synthesis by platelets: historical and new perspectives. J Thromb Haemost. 2009; 7 (2): 241-246.
7. Adams D, Gonzalez-Duarte A, O'Riordan W D, et al. Patisiran, an RNAi Therapeutic, for Hereditary Transthyretin Amyloidosis. N Engl J Med. 2018; 379 (1): 11-21.
8. Devine DV, Serrano K. The platelet storage lesion. Clin Lab Med. 2010; 30 (2): 475-487.
9. Bynum J A, Meledeo M A, Peltier G C, et al. Evaluation of a lyophilized platelet-derived hemostatic product. Transfusion. 2019; 59 (S2): 1490-1498.
10. Kulkarni J A, Cullis P R, van der Meel R. Lipid Nanoparticles Enabling Gene Therapies: From Concepts to Clinical Utility. Nucleic Acid Ther. 2018; 28 (3): 146-157.
11. Levin E, Culibrk B, Gyongyossy-Issa M I, et al. Implementation of buffy coat platelet component production: comparison to platelet-rich plasma platelet production. Transfusion. 2008; 48 (11): 2331-2337.

1. A lipid nanoparticle (LNP) composition for expression of exogenous protein in a population of transfected platelet cells, said LNP composition comprising a messenger RNA (mRNA) encoding the exogenous protein and a lipid mixture, said lipid mixture comprising:
a. at least one (ionizable) cationic lipid;
b. at least one helper lipid;
c. a sterol; and
d. a least one lipid-polyethylene glycol conjugate.

2. The LNP composition of claim 1, the lipid mixture comprising,
a. 30-55 mol % of the at least one (ionizable) cationic lipid
b. 5-20 mol % of the at least one helper lipid;
c. 25-50 mol % of sterol; and
d. 0.5-3 mol % of the at least one lipid-polyethylene glycol conjugate.

3. The LNP composition of claim 1, the lipid mixture comprising,
a. 50 mol % of the at least one (ionizable) cationic lipid
b. 10 mol % of the at least one helper lipid;
c. 38.5 mol % of sterol; and
d. 1.5 mol % of the at least one lipid-polyethylene glycol conjugate.

4. The LNP composition of any of claims 1 through 3, wherein the at least one ionizable cationic lipid in the lipid mixture is CL4H6, SM-102, ALC-0315, CL1H6, CL15H6, CL1D6, or ALC-0159 or any combination thereof.

5. The LNP composition of any of claims 1 through 3, wherein the at least one ionizable cationic lipid in the lipid mixture is CL4H6 or ALC-0315.

6. The LNP composition of any of claims 1 through 3, wherein the at least one ionizable cationic lipid in the lipid mixture is CL4H6.

7. The LNP composition of any one of claims 1 through 6, wherein the at least one helper lipid in the lipid mixture is phosphatidylcholine (POPC), 1,2-dioleoyl-sn-glycero-3-phosphocholine (DOPC), 1,2-distearoyl-sn-glycero-3-phosphocholine (DSPC), 1,2-dioleoyl-sn-glycero-3-phosphoethanolamine (DOPE), dioleoyl phosphatidylglycerol (DOPG), egg sphingomyelin (ESM), or any combination thereof.

8. The LNP composition of any one of claims 1 through 6, where the at least one helper lipid in the lipid mixture is phosphatidylcholine (POPC), 1,2-Dioleoyl-sn-glycero-3-phosphocholine (DOPC), or a combination thereof.

9. The LNP composition any one of claims 1 through 6, where the at least one helper lipid in the lipid mixture is phosphatidylcholine (POPC).

10. The LNP composition of any of claims 1 through 3, wherein the at least one ionizable cationic lipid in the lipid mixture is CL4H6 and the at least one helper lipid in the lipid mixture is phosphatidylcholine (POPC).

11. The LNP composition of any of claims 1 through 3, wherein the at least one ionizable cationic lipid in the lipid mixture is ALC-0315 and the at least one helper lipid in the lipid mixture is 1,2-dioleoyl-sn-glycero-3-phosphocholine (DOPC).

12. The LNP composition of any one of claims 1 through 11, wherein the sterol in the lipid mixture is cholesterol or a cholesterol derivative, or a combination thereof.

13. The LNP composition of claim 12, wherein the sterol in the cholesterol derivative is cholestanol, cholestanone, cholestenone, coprostanol, cholesteryl-2′-hydroxyethyl ether, cholesteryl-4′-hydroxybutyl ether, beta-sitosterol, fucosterol, or any combination thereof.

14. The LNP composition of any one of claims 1 through 13, wherein the at least one lipid-polyethylene glycol conjugate is 1,2-dimyristoyl-rac-glycero-3-methoxypolyethylene glycol-2000 (DMG-PEG2000), 1,2-distearoyl-sn-glycero-3-phosphoethanolamine-N-amino (polyethylene glycol)-2000 (DSPE-PEG2000), or PEG-1,2-distearoyl-rac-glycero-3-methylpolyoxyethylene 2000 (DSG-PEG).

15. The LNP composition of any one of claims 1 through 13, wherein the at least one lipid-polyethylene glycol conjugate is 1,2-dimyristoyl-rac-glycero-3-methoxypolyethylene glycol-2000 (DMG-PEG2000).

16. The LNP composition of any one of claims 1 through 13, wherein the exogenous protein encoded by the mRNA is selected from the group consisting of: a coagulation factor, an anti-fibrinolytic protein, an anticoagulant, a fibrinolytic protein, an antimicrobial protein, an immunomodulator, an anti-cancer protein, a genetic editing protein, and a reporter protein.

17. The LNP composition of any one of claims 1 through 10, wherein the mRNA comprises one or more modified nucleotide.

18. The LNP composition of claim 11, wherein the nucleotide modification is 5-methoxyuridine, pseudouridine, or N1-methylpseudouridine.

19. Use of the LNP composition of any one of claims 1 through 18 for transfection of platelet cells with mRNA encoding an exogenous protein and expression of said exogenous protein in resulting transfected platelets.

20. Use of the LNP composition of claim 10 for transfection of platelet cells with mRNA encoding an exogenous protein and expression of said exogenous protein in resulting transfected platelets, wherein the resulting transfected platelets exhibit minimal activation.

21. A population of platelet cells expressing an exogenous protein, wherein said population of platelets is transfected with the LNP composition of any one of claims 1 through 18.

22. A population of platelet cells expressing an exogenous protein, wherein said population of platelets is transfected with the LNP composition of claim 10, wherein said population of platelets exhibits minimal activation.
